(19) United States
(12) Patent Application Publication
(10) Pub. No.: US 2022/0254501 A1
(43) Pub. Date: Aug. 11, 2022

(54) Comprehensive Pet Health Care System

(71) Applicant: Vet24seven Inc., Mountain View, CA (US)

(72) Inventors: Calbert Lai, Seattle, WA (US); Pamela Seavey, Seattle, WA (US); Peter Elkin, Los Angeles, CA (US); Laura Berg, Fountain Valley, CA (US); Crissy Allstott, San Antonio, TX (US); Ross Wright, Centerville, TX (US); Chaz Trapolino, Centerville, TX (US); John Januskey, College Station, TX (US); Melissa Fuller, College Station, TX (US); Sam Hooker, Ladera Ranch, CA (US)

(21) Appl. No.: 17/669,722

(22) Filed: Feb. 11, 2022

Related U.S. Application Data
(60) Provisional application No. 63/148,516, filed on Feb. 11, 2021.

(51) Int. Cl. G16H 50/30; A47F 10/02; G06Q 20/14

(52) U.S. Cl. CPC G16H 50/30; G06Q 20/14; A47F 10/02

(57) ABSTRACT

A method for providing pet related services to a user includes payment by the user into an accrual account. Pet health can be evaluated by both a veterinarian and machine system. Following confirmation of veterinary emergency visit by the user and pet, payment from the accrual account for a veterinary emergency visit can be made.

### RELATED APPLICATION

The present disclosure is part of a non-provisional patent application claiming the priority benefit of U.S. Patent Application No. 63/148,516, filed on Feb. 11, 2021, which is incorporated by reference in its entirety.

### FIELD OF THE INVENTION

This invention relates generally to the field of animal services, including pet related healthcare and payment systems for veterinary consultations.

### BACKGROUND OF THE INVENTION

Some of the largest consumer and commercial market segments relate to animal management, feeding, and care, including veterinary services. Pet owners, livestock managers, or zoo personnel can all benefit from knowledgeable selection of food, medicine, or animal related products, timely delivery products or services, and an efficient history, monitoring, or tracking system. When an animal requires medical attention, a simplified system for access to a qualified veterinarian or animal professional for evaluation and treatment is needed. Such a system can include improved payment protocols to ensure access to a veterinarian or animal professional.

### BRIEF DESCRIPTION OF THE DRAWINGS

The specific features, aspects and advantages of the present invention will become better understood with regard to the following description and accompanying drawings where:

FIG. 1 illustrates a comprehensive animal care system that can include a voice responsive device;

FIG. 2A illustrates use of a voice responsive animal care system to support user interactions with a machine script;

FIG. 2B illustrates use of a voice responsive animal care system to support conversational user interactions that result in receipt of a product or service;

FIG. 2C illustrates use of a voice responsive animal care system to support user interactions that result in a veterinary consult;

FIG. 2D illustrates use of a voice responsive animal care system to support user interactions that result in interaction with other users;

FIG. 2E illustrates use of a voice responsive animal care system to support short term safety monitoring of household pets;

FIG. 2F illustrates use of a voice responsive animal care system to support long term health tracking;

FIG. 2G illustrates use of a comprehensive animal care system with support for payment services; and

FIG. 2H illustrates use of a comprehensive animal care system including user input processed for machine intelligence evaluation.

### DETAILED DESCRIPTION

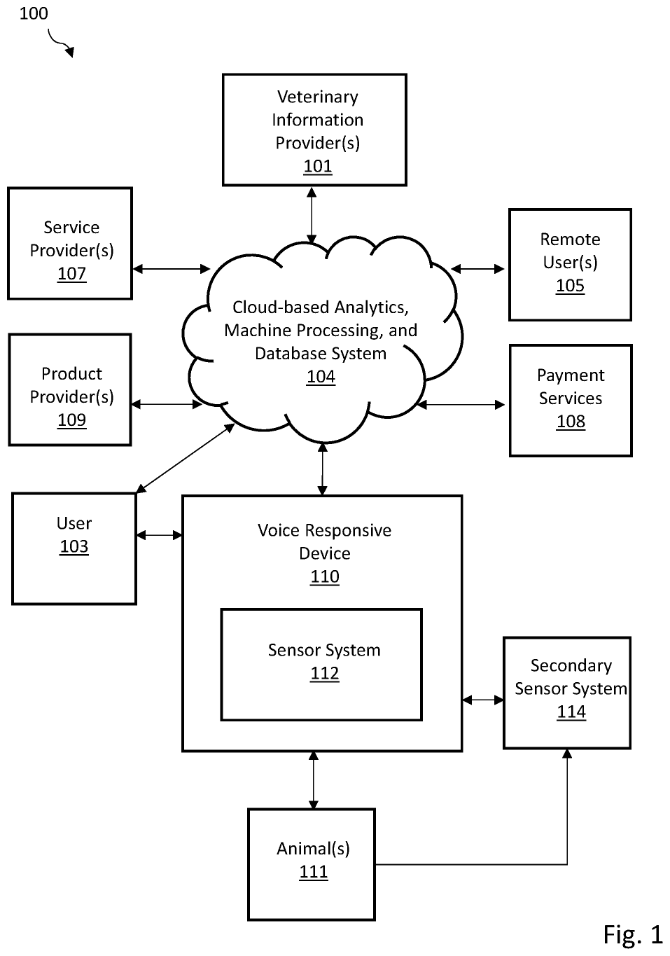

FIG. 1 illustrates a comprehensive animal care system 100 that can optionally use one or more voice responsive devices 110. The voice responsive device 110 can include an on-board sensor system 112 and/or a remotely located secondary sensor system 114. The voice responsive device 110 can be connected to cloud-based analytics, machine processing, and database system 104 that is able to provide various automated services. In addition to the automated services, the cloud-based system 104 can support interactions by a user 103 with other persons or organizations, including veterinary information providers 101, remote users 105, service providers 107, payment services 108, and product providers 109. Interactions between user 103 and the cloud-based system 104 can be mediated through voice responsive devices 110, or alternatively or in addition through keyboard, touch, mouse selection, or menu interaction.

The voice responsive device 110 can include custom audio and non-audio sensors, as well as voice identification and recognition hardware/software configured for providing animal related information, services or products. In some embodiments, the voice responsive device 110 can based on commercially available devices. Voice controlled personal assistant systems are widely used as part of fixed or mobile devices, or as an independent application (“app”), as software programs, or as operating system user interface modules for a smartphone, laptop, or other type of computer. Voice recognition, interpretation and response can be done locally, or as part of the cloud-based system 104 that receives filtered and compressed voice or sound recordings, analyzes the recordings, and takes needed actions. Voice synthesis can be used to provide automated spoken replies that can be streamed back to a local device.

In some embodiments, the voice responsive device 110 can include a voice activity detection module to distinguish voice from environmental or animal sounds, a beam forming module to provide a single voice stream derived from multiple microphones, a noise reduction module, a key word detection module, and a command phrase detection module. For example, in situations such as use of system 100 in an animal feedlot, multiple microphones may be needed to adequately cover a noisy area with substantial animal sounds. Voice commands need to be distinguished from animal sounds, environmental noise reduced, and a single voice stream can be used to recognize key words and command phrases. In some embodiments, the voice activity module can be additionally configured to detect a particular animal sound activity (e.g. specific animal distress sounds).

The sensor system 112 includes on-board sensors that are physically adjacent or attached to the voice responsive device 110. The secondary sensor system 114 can be remotely located, but capable of permanent or transient wireless or wired communication with the voice responsive device 110. The sensor system 112 and secondary sensor system 114 can include, but are not limited to, audio microphones, infrasound or ultrasound detectors, air temperature, air pressure, humidity monitors, accelerometers, physical pressure sensors, or position sensors. Video or still camera systems with face recognition capabilities, animal recognition, or other identification, tracking, or object count support can be used. In some embodiments, secondary sensor system 114 can include active animal tag systems that are implantable, attachable to an ear or other animal part, or worn on a collar or harness. These systems can provide a wide range of animal physiological information, including body temperature, pulse rate, oximetry, or animal breathing sounds.

In one aspect, sensor system 112 form a part of mobile devices with video or still camera systems and audio/video (A/V) communication capabilities, such as, for example, smart phones are used to connect animals and veterinarians in ways other than physical proximity. Using a mobile device's camera to take photos and/or video and send those to a veterinarian permits greater degree of detail to be communicated in triaging or diagnosing or follow-up for an animal. Additionally, a camera can be used to conduct a video chat between an animal owner and a veterinarian allowing real-time viewing and conversation between the two parties.

Cloud database 104 can include multiple databases and multiple interfaces. Some interfaces may be optimized for providing response to non-veterinary professional user requests, while other interfaces can be designed for effective use by livestock handlers or veterinary professionals. The cloud database 104 can be capable of storing a wide range of historical service, product, or sensor data (including animal audio related data such as individual sonograms). The cloud database can support manual, semi-automated, or fully automated access. In certain embodiments, standardized formats for veterinary information can be used, simplifying interchange of animal related information between veterinary professionals.

The veterinary information provider can be a local, national, or international business or organization capable of providing a range of animal related information available on a cloud database 104. In addition to factual information about animal and animal health conditions, veterinarian information provider data can include data about the veterinarian or veterinary professional. This information can include type of practice, such as, for example, single or multiple veterinarian, or association with a clinic. Veterinarian data can also include practice name, practice address, practice phone number, indication of single or multiple veterinarian practice. For each veterinarian associated with a practice, veterinarian data can include a name, email, phone number, species treated, hours of availability, academic credentials, specialties (e.g., board certified), a photo, a personal statement, licenses, tax forms, and direct deposit information. Veterinarians can use a veterinarian application to register with the network-based veterinary service provider. Veterinarian registration can include submitting veterinarian data, such as, for example, name, address, phone number, single or multiple veterinarian practice, species treated, credentials, any specialties, licenses, direct deposit information (for receiving payments), clinic information when applicable, a personal statement, etc. Veterinarian data can be stored in a veterinarian database and be made accessible to persons interacting with the voice responsive animal care system 100 or similar network-based veterinary service provider database.

First and second users 103 and 105 are representative, and can include single users, multiple users in a same family, organization, or business, or unrelated users. Users can be in geographically near or remote association. In some embodiments, actual identification and verification of particular users is necessary for access to premium services or to allow for payment for products. Such identification can occur through enrollment in services or systems associated with voice responsive device 110, or can be through separate fingerprint, voiceprint, other biometric services. Password or cryptographic token-based services can also be used. In addition to providing data via a voice responsive device, a user 103 can use a client application to register with the voice responsive animal care system 100 or similar network-based veterinary service provider. Registration can include submitting user 103 data, such as, for example, contact information (e.g., name, mailing address, email, etc.), username, password, billing information (e.g., credit card information), associations with any veterinary clinics, etc. and agreeing to terms of service. User 103 can also setup animal profiles for one or more animals, including animal profile information (e.g., name, age, specifies, breed, genera, photo, etc.). User 103 data, including animal profiles, can be stored in a cloud or user database and be made accessible to persons interacting with the voice responsive animal care system 100 or similar network-based veterinary service provider database.

Service providers 107 can include any animal related service, including but not limited to animal purchase, sale, adoption, feeding, pet sitting, grooming, veterinary services, or animal related information.

Payment services 108 can take advantage of connection to the voice responsive device 108, or optionally can work independently of voice responsive device 108 by using conventional keyboard or selection menu interface available on personal computers, tablets, or smart phones. Payment services can include support for pet insurance, pre-payment for veterinary services, or credit based payment methods.

Product providers 109 can include any animal related product, including but not limited to animal food, vaccines or medicine, grooming products, or animal toys. Product providers can be online or brick and mortar businesses. Retail, wholesale, or co-op businesses can provide products by direct shipping, mail, parcel delivery services, shipment to a site, by holding for pick-up, or by providing notice of likely or certain store availability.

Animals 111 can be any of a wide range of mammalian, avian, reptilian, amphibian, piscine, or invertebrate animals. Typical pet animals include dogs, cats, horses, rabbits, parrots, snakes, frogs, tropical fish, and tarantulas. Animals typically maintained as livestock can include cattle, pigs, sheep, goats, rabbits, turkeys, or chickens.

Aspects of the system and method can be appreciated with reference to the following exemplary scenarios:

Scenario 1—User interactions with Machine Sub-System

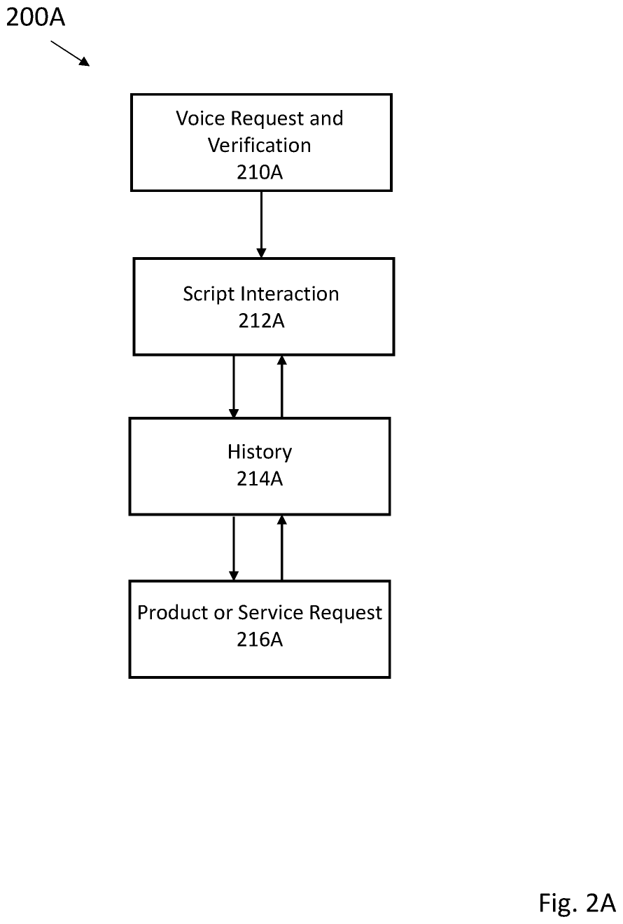

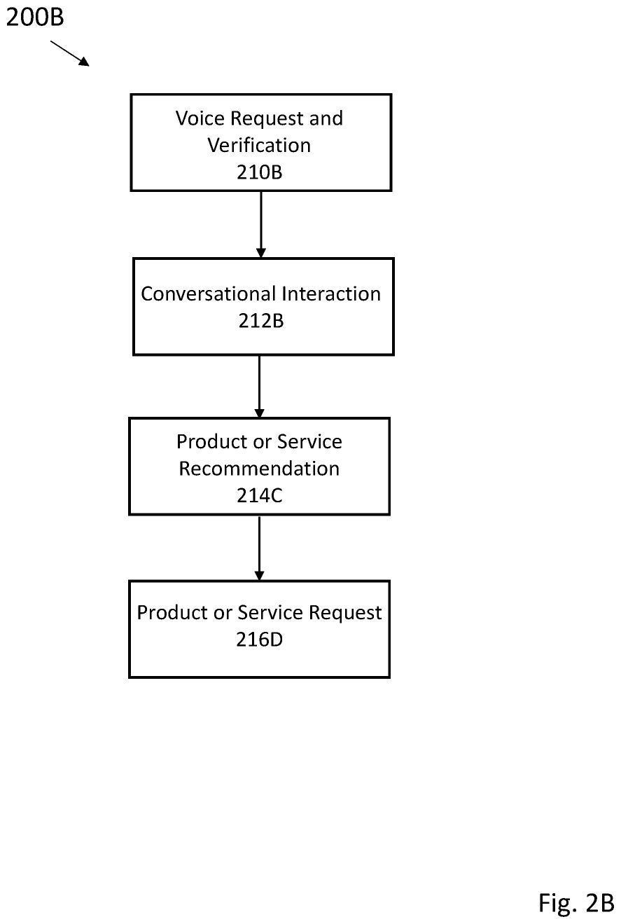

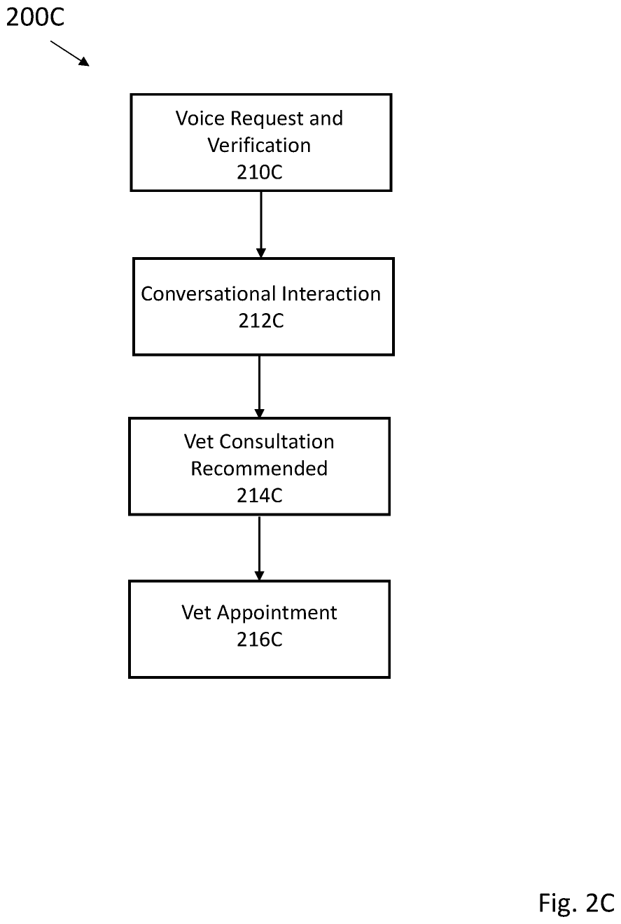

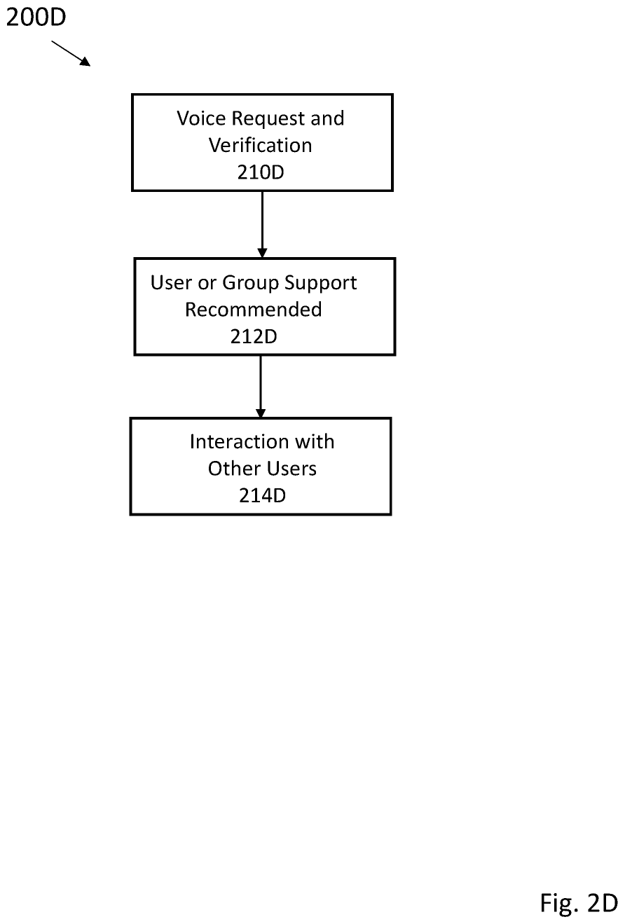

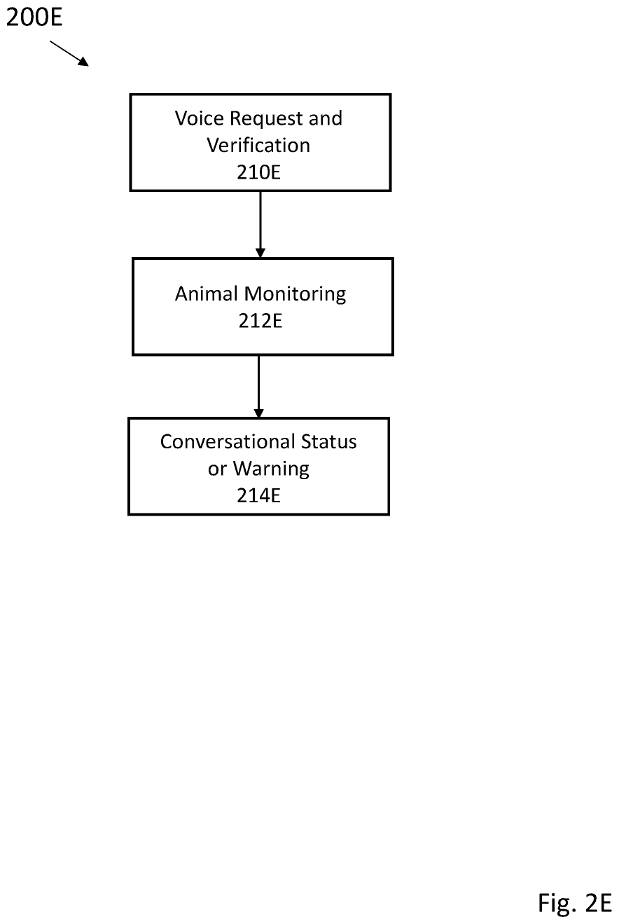

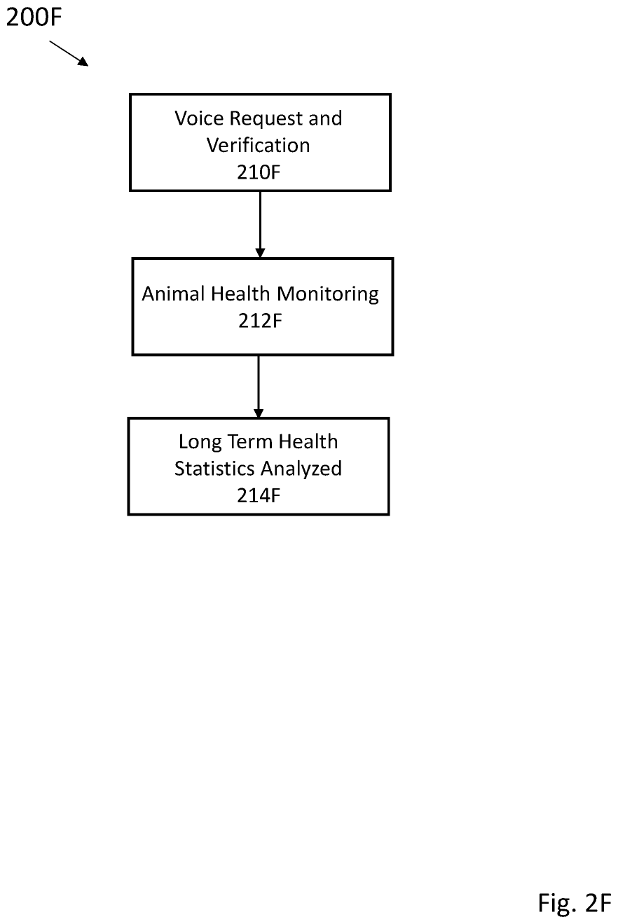

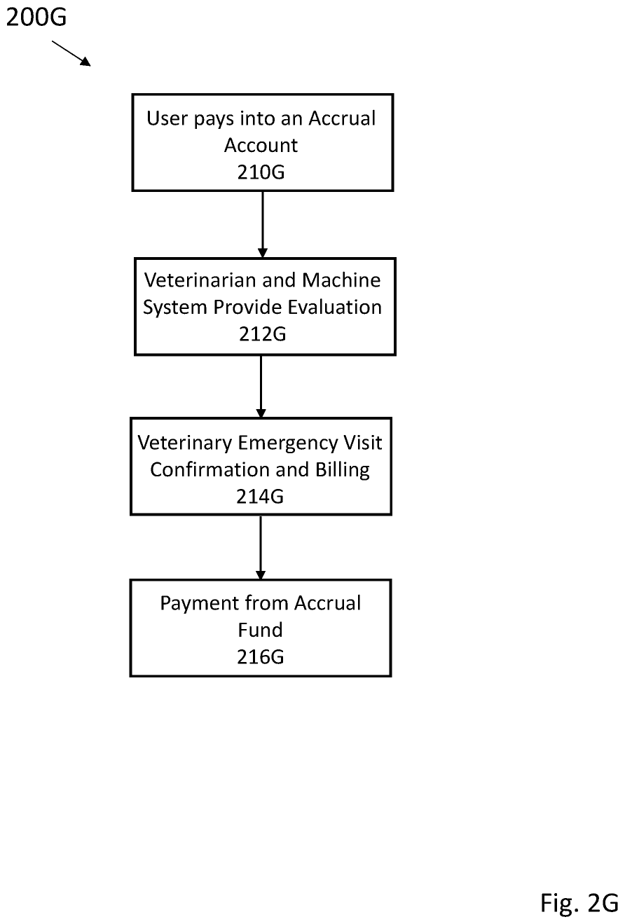

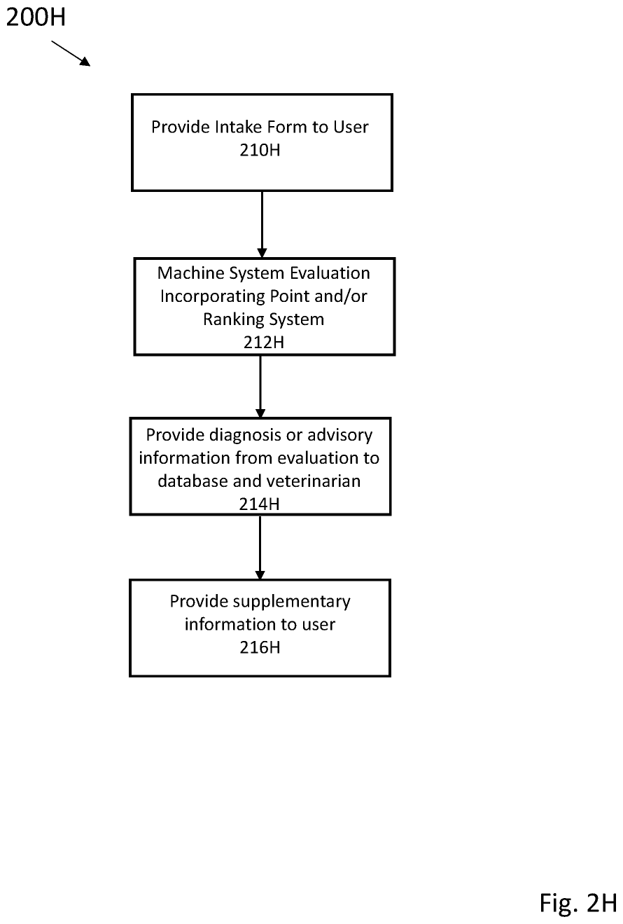

In one embodiment, a user 103 can require advice with respect to behavior or health conditions of an animal. Instead of needing assistance from a live veterinarians or animal care professionals, the system 100 of FIG. 1 can be used. As seen with respect to FIG. 2A, a verified voice request 210A can be made for animal related information. Verification can include but is not limited to voice identity recognition, verification via association with a particular device, biometric identification or voice passwords. In some embodiments, verification is needed before access to certain services or ability to purchase products is allowed.

Following verification, a scripted, expert system, or machine intelligence directed interaction 212A with a cloud-based voice recognition system can occur. Reference can be made to previous scripted interactions and new information can be stored by history module 214A. Based on history or scripted results, recommendations for product purchase or service requests 216A can be made.

Conversational style scripted interactions can be used to provide information with or without a request for services or products. Scripted interactions can be used for animal care emergency triage, or to help direct users to needed products or services.

As an example, a script interaction for feline emergency care can proceed in a manner illustrated as follows:
START
USER: My Cat is vomiting what do I do?
SYSTEM: I am sorry to hear that your cat is vomiting. What is your cat's name? USER: <Fluffy>
SYSTEM: I need to ask you some questions about <Fluffy>. Is <Fluffy> vomiting blood?
USER: No
SYSTEM: Has <Fluffy> eaten anything like part of a toy, string, fabric, something rubber, plastic or something else that could block the intestinal tract?
USER: No
SYSTEM: Could <Fluffy> have gotten into any human food or human medications?
USER: No
SYSTEM: Has <Fluffy> had any recent changes in diet or treats like flavor, brand or type?
USER: Yes
SYSTEM: It is possible that <Fluffy> may have a sensitivity to something in the new treat or food. Ideally food changes should be made slowly over a 21-day period. If you would like to speak with a veterinarian now to explore this further and to get some helpful hints, I can connect you with a live veterinarian now. Would you like to talk with a veterinarian now? Please answer Yes or No.
USER: Yes
GOTO CLOSE SUBTREE

In an alternative scenario where the answer to the SYSTEM question “Has <Fluffy> had any recent changes in diet or treats like flavor, brand or type?” is “No”, the following alternative script can be used:
SYSTEM: Has <Fluffy> had any recent changes in diet or treats like flavor, brand or type?
USER: Yes
SYSTEM: It is possible that <Fluffy> may have a metabolic issue—this refers to a complex of disease processes that are associated with the liver, kidney, pancreas, or the endocrine system (pituitary, thyroid, adrenal glands, ovaries testes). If you would like to speak with a veterinarian now to explore this further and to get some helpful hints, I can connect you with a live veterinarian now. Would you like to talk with a veterinarian now? Please answer Yes or No.
USER: Yes
GOTO CLOSE SUBTREE
   Scenario 2—User interactions with Service or Product Providers

In one embodiment, a user 103 can require provision of grooming or other services and/or products for an animal. The system 100 of FIG. 1 can be used provide such services or goods. As seen with respect to flowchart 200B FIG. 2B, a verified voice request 210B can be made for animal related product or services. Following verification, a conversational interaction 212B with a person, a script, an expert system, or a machine intelligence can occur. Based on history or conversation results, recommendations for product purchase or services 214C can be made. If the recommendation is accepted by the user 103, a product purchase or services request 216D can be made. These can include, but are not limited to products such as food, grooming products, pet toys, medicine or supplements, and services such as pet sitting or walking services, grooming services, and feeding or health monitoring.

Scenario 3—User interactions with Veterinary Service Providers

In one embodiment, a user 103 can require provision of grooming or other services and/or products for an animal. The system 100 of FIG. 1 can be used provide such services or goods. As seen with respect to flowchart 200C of FIG. 2C, a verified voice request 210C can be made for animal related product or services. Following verification, a conversational interaction 212C with a person, a script, an expert system, or a machine intelligence can occur. Based on history or conversation results, veterinary consultation recommendation 214C can be made. If the recommendation is accepted by the user 103, a veterinary appointment 216C can be made.

In certain embodiments, veterinarian consults requests can be made to veterinarians suitable to handle the veterinary consults. A consult module can receive veterinary consult requests from users of the voice responsive animal care system 100. A veterinary consult request can include consult data defining the parameters of the veterinary consult, such as, for example, a species, urgency, and preferred communication mechanism. Consult module can refer to user 103 data and to veterinarian data for registered veterinarians of the voice responsive animal care system 100 or similar network-based veterinary service provider. Based on the consult data, the user 103 data (including an animal profile), and the veterinarian data for registered veterinarians, the consult module can identify one or more veterinarians suited to handle the veterinary consult request. One of the suitable veterinarians can then accept the request and perform the veterinary consult via the preferred communication mechanism. Once a veterinary consult is accepted, communications between a veterinarian and a user 103 can be established with the same the voice responsive device 110 used to interface with network-based veterinary service provider or alternatively, can be established with different devices.

Typically, a user 103 provides a voice request for a veterinary consult to provide medical analysis with respect to a condition of animal. The user 103 can verbally relates species, animal name, animal symptoms, urgency, and communication type. Species can indicate the species (e.g., dog, cat, bird, etc.) of the animal. Urgency can indicate a time frame for when user 103 desires the veterinary consult to occur, such as, for example, in the next 15 minutes, within 4 hours, sometime today, within a day, etc. Communication type indicates the type of communication preferred by user 103, such as, for example, in-person visits to a veterinarian or animal health professional, in-person visits by a veterinarian or animal health professional, text messages, text chat, audio, audio/video, etc., when conducting the veterinary consult.

In one aspect, user 103 also includes one or more pictures and/or one or more videos of animal representative the animal 111's condition.

In another aspect, user 103 also describes animal 111's condition in one or more text fields. The one or more text fields can include a shorter text field configure to accept 2-3 words describing the animal's condition. The one or more text fields can also include a longer text field configured to accept potentially unlimited text used for a more detailed description of animal 132's condition.

In some embodiments, user 103 data can be provided to and from the cloud database 104. At least one of one or more veterinarians or veterinary professionals can be provided with the veterinary consult request. The consult request can include data provided by that user 103, including but not limited to animal species, the urgency, the preferred communication type, the user data, and any available third party or historical veterinarian data.

In one aspect, prior to notifying the one or more veterinarians a pricing matrix is used to determine a fee for the requested consult. The consult module presents the fee for approval by user 103. If user 103 approves the fee, processing of the consult request continues. Upon approval, the consult module can also pre-authorize a charge to a credit card or other payment mechanism. If user 103 does not approve the fee, processing of the consult request can be terminated. The price presented to a user 103 can be a factor of a pricing matrix, business or operating hours, and method (e.g., live video or chat (messaging). For example, an urgent, live video consult during the day may be priced differently than an urgent, live video consult in the middle of the night.

In one aspect, a veterinarian includes a proposed time to conduct the veterinary consult in consult acceptance. The proposed time can be included in acceptance notification. User 103 can agree to the proposed time or propose a different time. Proposed times can be exchanged until user 103 and veterinarian agree to a time.

During the veterinary consult, additional photos and videos can be transferred form user 103 to the veterinarian or veterinary professional. Prescriptions for animal can be emailed or otherwise communicated to designated recipients including the user 103, a pharmacy, or another person. A summary of the veterinary consult to user 103 and a record of the veterinary consult in added to the file for animal in the cloud database 104.

In one aspect, veterinary consults are conducted via live video or chat (messaging) within the user 103 and veterinarian application. Veterinary consults can be initiated by the veterinarian. When using chat to conduct a veterinary consult, the user 103 and veterinarian are notified when the other party sends a message. To conduct a veterinary consult via video, the veterinarian launches video chat and an invitation to join the video is sent to the user 103 via text message and in-app message. If the user 103 responds to the video invitation, the veterinarian connects with user 103. If the user 103 does not accept the video invitation, the user 103 receives a notification, and the veterinarian can attempt to contact the user 103 again. The video can have an indicator of signal strength and allow sharing of and drawing on photos. More than two individuals can participate in a live video or chat session. The veterinarian and/or user 103 can invite additional participants. For example, a veterinarian can invite another veterinary, for example, a specialist, into a live video or chat session. A user 103 can invite another person interested in the care of the animal, for example, a family member, into a live video or chat session.

In aspects, a user 103 may have a preferred veterinarian. The consult module can direct the user 103′s veterinary consults to the preferred veterinarian when available. When a preferred veterinarian is not available, the consult module can provide the user 103 with alternate veterinarians capable of handling a particular veterinary consult. In a multiple veterinarian practice, alternate veterinarian can be another veterinarian in the same practice. When no other veterinaries are available from the same practice (e.g., when a clinic is closed), the consult module can identify other alternate veterinarians. An alternate veterinarian can be selected based on one or more of: geographic location, licensure, availability, ratings, number of consults performed, species treated, as well as any other information contained in a veterinarian or practice profile.

When the veterinary consult is complete, the payment information for the user 103 is used to bill the user 103 a specified fee. A portion of the specified fee can be retained by the network-based veterinary service provider in exchange for use of a voice responsive user interface platform. The remainder of the specified fee is remitted to the veterinarian, for example, using the direct deposit information.

After a veterinary consult is complete, the user 103 and the veterinarian are provided an option to rate one another.

Scenario 4—User Interactions with Other Remote Users

In one embodiment, a user 103 can interact with other users 105, including animal enthusiasts or appreciation groups, animal advice providers, breeders, or experts on various products and services. The system 100 of FIG. 1 can be used to provide such interactions. As seen with respect to FIG. 2D, a user can make a voice request and receive verification 210D, find (212D) a recommended user or group for support via machine or script recommendation, and interact (214D) with those users. Animal related social interactions can involve contacting skilled non-professionals with an interest in conversation, tips on animal handling, or recommendations or references. For example, a user interested in acquiring a particular animal breed can talk with a remote user and receive advice and recommendations for local breeders.

### Scenario 5—Short Term Safety Monitoring of Household Pets

In one embodiment, a user 103 can monitor animal location, sounds, interaction with internet connected toys, food and drink consumption, or animal health status information. The system 100 of FIG. 1 can be used to provide such interactions. As seen with respect to FIG. 2E, a voice responsive device 110 with onboard sensors 112 and optionally associated secondary sensor systems 114 can be used for animal sound monitoring. A user can make a voice request to begin monitoring with various set parameters (e.g. do not monitor when people are in the house) and receive verification (210E), let the voice responsive system monitor animal sounds and other sensed features (212E), and receive conversational warnings with respect to the animals if needed (214E). Monitoring can include individual animal sound and voice recognition. Specific location can be monitored generally, and specifically within a known structure by monitoring the position of an animal-attached device relative to known placements of structure-connected partition transmitters and sensors within a home or other structure, as well as intra-animal interactions (e.g. detected cat fights). Remote detection of animal location, door positions (open or closed), and other features relative to the structure and position of the animal can be controlled or altered remotely.

### Scenario 6—Long Term Health Tracking

In one embodiment, a user 103 can allow long term monitoring of animal health status information such as weight, sleep, or activity patterns. The system 100 of FIG. 1 can be used to provide such interactions. As seen with respect to FIG. 2F, a voice responsive device 110 with onboard sensors 112 and optionally associated secondary sensor systems 114 can be used for animal sound monitoring. A user can make a voice request to begin long term health monitoring with various set parameters (e.g. determine animal sleep cycles) and receive verification (210F), let the voice responsive system monitor animal activities and other sensed features (212F), and receive long term health statistics and optional related advice (214E). Monitoring can include individual animal sound recognition relevant to movement, activities, or vocal output that can be used to determine the health, stress levels, and other health parameters of the animal.

### Scenario 7—Payment Services

In one embodiment, as seen with respect to FIG. 1 and process 200G of FIG. 2G, a user 103 can take advantage of various payment mechanisms using payment services 108. For example, a user 103 can be a member of a rainy day fund that can be used to pay for emergency veterinary treatment. In one embodiment, a user 103 pays in an accrual account (210G). If a pet emergency occurs, voice or electronic contact with a veterinarian is made. Using cloud-based analytics, machine processing, and database system 104 such as discussed with respect to FIG. 1, a veterinarian uses the received information to provide evaluation and referral for emergency veterinary treatment (212G). The received information can include prior pet health history, preliminary machine diagnosis, or machine provided advisory information. After a veterinary emergency visit confirmation and billing is received (214G), payment from accrual account can be made. In some embodiments, confirmation, billing, and payment authorization can be made using one or more voice responsive devices, while in other embodiments keyboard, touch, mouse selection, or menu interaction can be used. In one embodiment, a method for providing pet related services to a user includes payment by the user into an accrual account. Pet health can be evaluated by both a veterinarian and machine system. Following confirmation of veterinary emergency visit by the user and pet, payment from the accrual account for an veterinary emergency visit can be made.

In more detail, one embodiment of an accrual account that pays for emergency veterinary treatment can proceed as follows:

Pet Owners (PO) pay into a fixed cap fund (for example, one of up to $1000) that accrues over 24 months and can be used to pay for Emergency and Emergency Room (ER) visits. A PO accrues an amount each month until reaching the fixed cap. If funds fall below the cap, PO's start accruing again until reaching the cap. As will be understood, in some embodiments the cap value can be adjustable, vary according to user membership or usage plan, or the cap can be removed.

### Rules

1. The accrual based Rainy Day Fund (RDF) is based at the PO account level and can be used for any pet in the account.

2. The fund has a cap over 24 months and an amount is added each month up to the cap. If RDF are used and drop below the cap, then accrual starts again.

3. There is a 7 day grace period from membership start where fund cannot be used by PO.

4. Any remaining annual membership amount is not removed when RDF is used.

5. PO must communicate with a Veterinarian who must assign the action item of “Refer to Emergency Clinic”.

6. PO must check-in at an ER within 10 hours of chat completion time.

7. If needed, help in finding an open ER facility can be provided.

8. RDF feature is available to all members who have purchased a product that includes the RDF feature or customers on co-marketing “limited time promotion” offers

### Process

1. When PO has a concern, they must communicate with a Veterinarian.

2. If Vet deems the situation an emergency they will select “Refer to Emergency Clinic” and transfer PO to Customer Care (CC)

3. CC will:
a. Confirm with PO the available amount of their RDF
b. Review with PO the RDF rules and process
c. Give PO the 800 number to call once ER visit is complete
i. Backup plan: If ER clinic will NOT do the phone call, the PO an email address will be provided to allow emailing ER documents and providing the ER with a credit card number.
d. The CC chat can appear in chat transcript so the PO can refer to details for processing payment.

### Verification/Payout

1. PO must check in at ER within 10 hours of chat completion

2. CC can pay the ER directly, 24/7

3. ER can phone CC to get payment. CC must determine if:

4. ER recommendation was made by an authorized Veterinarian

5. It is the correct PO & Pet Name

6. Diagnosis matches chat

7. Check-in time at ER was within 10 hours of chat conclusion

8. ER is a valid location

9. How much money to pay out: Up to their RDF balance

10. CC pays ER via credit card over phone and payment from the RDF is recorded.

For those situations when an ER is unable to phone CC, the PO can email CC the ER docs and CC calls ER.

### RDF Balance/Payouts

1. RDF overview and rules can be provided to a PO.

2. RDF balance and/or balance history can be graphically displayed.

3. At payout, CC can review PO RDF balance in order to determine RDF payout.

4. When a payout from RDF is made, the PO account accessible via app can reflect payout and new balance. Fields shown can include date of payout, starting balance, payout amount, new balance, veterinarian/ER Name paid, and Pet Name.

5. An email can be triggered when PO uses RDF and can reference date of payout, starting balance, payout amount, new balance, veterinarian/ER Name paid, and Pet Name.

### Scenario 7—User Provided Intake Information

In one embodiment, as seen with respect to FIG. 1 and process 200G of FIG. 2H, a user 103 can use an intake form (210H) completed in response to voiced questions or directly filled using keyboard or other writing input interface. The information from the intake form can provide ranking or numeric points that be used by cloud-based analytics, machine processing, and database system 104 such as discussed with respect to FIG. 1 (step 212H). A veterinarian can use the received intake form information and any machine processed information derived at least in part from the intake form to provide evaluation and referral for veterinary treatment (212H). The received information can include prior pet health history, preliminary machine diagnosis, or machine provided advisory information (214H). If needed, supplementary information can be provided to the user 103 (step 216H), including diagnosis, product, marketing, or payment information.

In more detail, one embodiment of an app based intake form that provides information to a user 103, the cloud-based analytics, machine processing, and database system 104, and authorized veterinary professionals can be outlined as follows:

App Intake Form Write-up (aka “quiz”)

Intro Screens (first time app download):

*Initial screen with pet illustration

Welcome 1:

Sit, stay, roll over.

Welcome 2:

Just kidding, you don't need to jump through hoops with us!

No tricks, just expert help and advice.

Tell us about your pet!

Welcome 3:

We take the guesswork out of pet parenting.

Got 3 minutes? We want to get to know you! Take our FREE pet wellness quiz now.

Quiz Intro Screen:

Take the first step to becoming the best pet parent you can be!

You can:

Chat with a licensed vet 24/7.

Save time and money with expert advice from home.

Rock pet parenting!

Get started with a trial offer: [Enter Trial Discount Code Here]

### >>Welcome COMPANY NAME Member!

Let's get started!

CTA 1: [Take the Quiz] (user continues in flow)

CTA 2: [Learn More First]—user goes to supplementary screen below

CTA 3: [Chat with a Vet] (I'll take the quiz later)

### >>Supplementary Screen for [Learn More First] CTA:

Did you know?

Daily pet care will keep your pet healthier, reduce the occurrence of chronic conditions and save you up to $700 a year in vet costs.

Save time, stress and money!

Our Members Get:
24/7 unlimited live consults with licensed vets, no appointments needed.
Rainy Day Fund with up to $1,000 to help with emergency vet visits.
Welcome bag with home lab testing kit included—plus product samples!
Pet Health Assessment to get you started on your pet wellness journey
Trial offer with no commitments, cancel anytime.

Ready for more frequent tail wagging? Get your free pet health assessment.

CTA 1: [Take the Quiz]

CTA 2: [Chat with Vet] (I'll take the quiz later)

Quiz Begins:

Q. We are lovers of all animals—what kind of pet are you parenting?
Dog
Cat
Other

Got a full house of furry friends? Choose one to start—don't worry we won't assume favorites—and add more later at no extra cost.

Woo hoo, dogs rule!

>>Supplementary Screen for Answers other than Dog—User then goes to Value Prop Screen on Page 4:

We Love PET TYPE

Our vets can consult on every type of animal—big, small, furry, scaly, feathered and more. Start a chat 24/7 with one of our licensed vets to get personalized advice and help on any pet.

Meanwhile, we've got all paws on deck as we continue to build out our pet wellness quiz to include all pets and meet all pet parents' unique needs.

Q. What's your pup's name?______

To get NAME the best care, we'll need your email address to stay in touch:

First Name ______

Last Name ______

Email ______

Thanks! You're less than 3 minutes—two quick rounds of fetch in dog speak—from finishing up. Just a few quick questions about you and NAME . . .

Let's get to know NAME!

He/She (dropdown) is ______ (fill in blank) years/months/weeks (dropdown).

He/she (autofills depending on the response) is/is not (dropdown) neutered and weighs about ______ (fill in blank) lbs

Look at The Farmers Dog example: https://www.thefarmersdog.com/signup/pets

Name is a ______ (breed dropdown with option to add additional breed for mix—is there a list of breeds we have that we can pull in?) and is the most ______ (dropdown) pooch.
Silly
Playful
Cuddly
Smart
Sneaky
Lazy

Q. Ok, now a little about you. How experienced are you as a pet parent ______ (dropdown)?

Help—first time parent here! +1

I've got some experience under my collar. +2

Not to scratch my own back, but I'm an old pro. +3

Q. My pet parenting style is ______ (dropdown)

Let's just say it's my dog's house, I just live in it. +2

Helicopter Parent here—I may be slightly over protective and watchful. +2

Somewhere in between. +3

Q. NAME and I like to (can choose more than one):
Walk +3
Run +3
Play fetch/games +3
Swim +3
Hike +3
Relax [symbol] +1
Cuddle +1
Other +2

### Second Level Question:

NAME and I do these activities ______ (dropdown)
Daily +4
Weekly +3
Monthly +2
Rarely −1

Note: Points on selected Questions's will be tallied and added to labeled topic to trigger topic where help is available. The points will also count towards the overall score.

Q: Does NAME have any ongoing issues (can choose more than one)?
Digestion −2 Nutrition
Skin irritation −2 Grooming/Skincare
Food allergies −1 Nutrition
Other allergies −1
Weight −3 Exercise+Nutrition
Urinary issues −2 Nutrition+Behavior
Injury −1
Bad breath −1 Nutrition
Anxiety −1 Anxiety
Other ______ −1
None

### Second Level Question:

NAME has/has not ______ (dropdown) experienced any of these health issues in the last six months.
Has −5
Has not +5

Q: NAME has/has not (dropdown) visited the vet in the last year?
Yes +4
No −4

Q: Just like any relationship, your pal probably has a few habits that drive you nuts! What do you wish NAME didn't do (can choose more than one)?
Scratch −1 Behavior
Jump −1 Behavior
Chew −2 Behavior
Have potty accidents −2 Behavior+Nutrition
Bark −1 Behavior
Bite/lick −2 Behavior
Shed −1 Grooming/Skincare
Other ______ −1

Q: How about things you'd love to see more of (can choose more than one)!
Cuddle −1
Play −2
Listen −3
Sleep −1
Exercise −3
Relax −1
Show affection −1
Other −1

Q: What main areas do you think NAME could use improvement on (can choose more than one)?
Nutrition −3 Nutrition
Weight −4 Nutrition+Exercise
Dental −4 Dental
Grooming/Skincare −2 Grooming/Skincare
Exercise −4 Exercise
Behavior −3 Behavior
Anxiety −2 Anxiety
Other −2

### Account Creation Screen:

Please set a password to complete your profile and see NAME' s assessment results!

Email: (autofilled) and/or ask for mobile number if need it

Password: ______

Verify Password: ______

### Results Screen:

Congratulations—and paw to the heart, you and NAME are a dynamic duo. With some expert advice from our vets, you two are unstoppable!

Here's where NAME placed on our pet health scoring system:

Visualization should be based on quartiles with an indicator showing where a PO landed post-quiz. Each quartile could be represented by a different color—we should use colors that match our branding (PO does not need to see the other quartiles)

While a member does not see the raw added numbers from the questions, four ranges based on projected results can be shown:
Green (score of 51 or higher): You're a pro! By working with our licensed veterinarians we can make sure NAME maintains optimal health.
Blue (score of 26-50): Looking good! With a few improvements NAME will be on their way to feeling their very best.
Marigold (score of 0-25): Let's work together! With guided advice from our vets we can get NAME feeling their best!
Teal (score of 0 or less): We'd love to help! Our vets are experts in all things petcare. When you have a question, we have the answer!
*Quartile placement: first (score of 0-25), second (score of 26-50), third (score of 51-75), top (score of 76-100)/scoring is based on total number of points divided by 100

Each membership comes with a complimentary lab testing kit—our vets will guide you through the results and show you how to use them to improve NAME' s health!

CTA: [Choose lab testing kit here]

Here are some key areas where we can help:

NOTE: These responses will be dynamically triggered based on quiz answers from red Qs. Categories with the most points will appear with a maximum of 2-3 categories being displayed on a single screen (descriptions would ideally “pop out” when tapped):

### Categories and Responses:

Nutrition: It's no surprise that the right food can make a huge difference in NAME's overall health. But, each pet is unique. Our vets will work with you to understand NAME's needs and preferences.
Dental: Did you know you should be addressing NAME's teeth X times per week? Without consistent care NAME may develop X, Y or Z. Our vets will guide you so you know just what to do and when!
Grooming/Skincare: Skincare issues can be tricky to spot and grooming advice can get confusing. We'll help you understand what NAME needs and recommend ongoing care to get them living their best life.
Exercise: Playtime is fun, but we also recognize it can be a commitment. Our vets will chat with you one on one to develop an exercise plan to keep you and NAME motivated!
Behavior: Wondering if something is normal? Not sure how to address it? Our vets have seen it all!
Anxiety: Sometimes it's difficult to understand how animals respond to stress. Our vets will work with you to recognize the behaviors and learn how to manage it.

Start your live vet chat now and take the next step to improving NAME's wellness today!

CTA [Get Started]

### Benefits Screen:

Your pet's health shouldn't start and stop with a vet visit.

Improve your pet's overall wellness while saving big bucks ($700/year on average)!

### Our Members Get:

24/7 unlimited live consults with licensed vets, no appointments needed.
Rainy Day Fund with up to $1,000 to help with emergency vet visits
Welcome bag with home lab testing kit included—plus product samples!
Pet Health Assessment to get you started on your pet wellness journey
Trial offer with no commitments, cancel anytime.

Checkout Screen:

Checkout Payment Options (TBD)

Apple Pay

Amazon Pay

Paypal

Credit Care

Do you have a coupon code? Enter it here ______ (hopefully we can auto apply it)

*Is your shipping address the same as your billing? {if no, then ask for it}—we're going to send you a treat!

[CONFIRM PAYMENT]

### Scenario 8—Care Coach Personal Guide

In one embodiment, operating as seen with respect to FIG. 1 and the disclosure herein, a Care Coach personal guide can be provided through an app or other user interface to the cloud-based analytics, machine processing, and database system 104. In one particular example, the Care Coach is a person with access to at least portions of the cloud-based analytics, machine processing, and database system 104 that can provide the following:

Answers to questions, needs for support, or to celebrate with members when their pets are getting healthier and happier. The Care Coach helps the member navigate the system by offering insights on how to access certain features and helpful hints. The Care Coach delivers surprise and delight to members with proactive communication when alerted of activities that would warrant a relationship building moment, by personalized communications both in-app and handwritten notes, as well as delivering digital engagement. The Care Coach can work to gather knowledge about the member in conversation for future reference for surprise and delight. The Care Coach's goal is to provide a fun, authentic, empowering, and supportive relationship with all members, providing value any time they can. A Care Coach will provide member satisfaction) and as a result, long-term retention (90+% over three years). Care Coaches can also be responsible for signing up members and retaining them throughout the lifetime of their pets. Care Coaches can be commissioned team members who will be paid upon acquisition and retention of members.

Member Functionality Requirements

Reaching the Care Coach

What Consumers does the Care Coach Support?

Members who have subscribed directly with us.

Pet Owners who have subscribed to premium features.

Messaging Feature In-App to Reach Care Coach

This feature requires the following functionality:

If member, messages route directly to the assigned Care Coach.

If a prospective member or registered user, they can click a button to Chat (with an available Care Coach) to get answers to questions about membership benefits and logistics. Care Coaches will engage with the prospective member to encourage them to sign up for a membership

When the messaging function is opened:

If Care Coach is ‘online’, a keyboard will pop up with the ability for a Member to write a message to their Care Coach

If Care Coach is ‘offline’, it will present a customized and personalized message from the Care Coach (see: Messaging), and options for other actions:

Want to touch base via phone? Schedule a future phone appointment with a Care Coach.

Will need a scheduling tool built or tied in, that the Care Coaches can modify based on their hours

Leave me a message! I′ll get back to you as soon as I′m back online!

Have the ability for Members to write a message that will be sent through anyway but will be later accessible for the Care Coach to respond when they return online.

Need help now? Route the incoming message to another Care Coach who is online.

Have a question for a Vet? Speak with a Vet right away for a Pet Health question.

Have a button to send them directly into Vet chat from this area within their app.

Allow visibility into chat history with Care Coach

If Care Coach communications span channels (moves from chat to phone, or email), have ability for a reference to those communications or a recap by the Care Coach.

Ability to send emojis, gifs, photos (heic, jpg, png, etc.), and PDFs

Care Coach Ratings

Ability to Rate and provide feedback on the Care Coach support (i.e. could be accomplished by a push notification asking ‘How am I doing?’ directly from the Care Coach)

Message Notifications for Member

When receiving a message, prospective member, registered user, or Member should receive a push notification and a visual indicator on the homescreen and the app icon that they have a message waiting.

Backend/Operations

Care Coach Backend Visibility includes the following information:

Member Details

Member Details

Location/Time Zone

Name

Email

Phone Number

Pet Details

Name

Age

Breed

Account Details

Account Status (Pre-Paywall/Active Member/Churned Member)

Account Creation Date

Membership Date (May 2019—CC Cancel Date (Past/Future)

Source Information (SPO or Member)

Rainy Day Fund

Amount Available

Log of Submissions, Approvals, Denials, and amounts

Any DNA/Labs Kits that were requested, or add-on products ordered

Tracking information

Status

Billing Details

Invoicing

Credit Card Expiration

Renewal/Cancel Date (Past/Future)

Communications

Asynchronous

Push

Email

Social Media

Chat History

Care Coach

Vet Chat

Support Email Inbox

Member Engagement

Quiz Answers and Scores

Member ratings and feedback

Content

Overall Content Library

Content that has been assigned to member

Care Coach Backend Functionality

Pre Paywall

When a prospective member downloads the app the Care Coach has the ability to reach out to them right away.

Have the ability to turn off responses to specific users (if users are taking advantage of Care Coach feature pre-paywall).

Communications

Messaging

Initiate messaging with a Member, even if the member does not reach out first.

Send and receive files to and from Members (photos, PDFs, etc.)

Send and receive gifs and emojis in chat tool

Be able to change the communication channel from messaging to another channel when requested.

If changing communication channels, have the ability for Care Coach to tie in the other communications.

If a chat moved to a phone call—have an area where Care Coach can log a phone call so the member can see the details on when they spoke.

Be able to leave a customized and personalized message when they are not on shift (i.e. Hi [Member Name]! I'm not available at the moment to chat, however, I will be back at 7 AM CST tomorrow (1/20) to check in. I can't wait to chat!)

Send Deep Links to allow member to go directly in to Vet Chat

Send URI to specific content

Message with Member in a tool outside of Async or Live Chat in Devon

For the long term solution, having a tool that is separate from Vet Chat experience would be best.

Have campaign functionality so Care Coach can send multiple personalized messages at one time (like anticipatory guidance, feature updates, etc.)

Push Notifications

Initiate a notification to a member (example: to notify them that lab tests results are complete and arrange a consultation with a vet)

Activity

Review all business data and activity for each/all member in one (or very few) place(s)

This is to include any brand communications, billing details, Care Coach comms, etc.

Content Assignment

Assign content to member library

Account Management

Cancel subscriptions and initiate refunds

Update any Member or Pet information on behalf of Pet Owner

Authorize/Decline Rainy Day Fund Requests

Review all member data and activity in one (or very few) place(s) for ease of support

Any vet chats, member acct info, demographic info, pet info, etc.

Communications

Review all business data and activity for each/all member in one (or very few) place(s)

This is to include any brand communications, billing details, Care Coach comms, etc.

If a Member comes through Vet chat with a question that should be routed to a Care Coach, Vet or shift lead has ability to route chat to Care Coach (later requirement)

Member Management

When a new member comes on board, auto-assign a Care Coach, based on a rotating schedule (future release)

Have a tool/place for Care Coach to keep notes and important dates for each member (i.e. member got pet when pet was 2 years old, member loves beach walks with pet, pet loves watermelon, members mom's dog and her dog are bonded, member loves photography or plays sports, etc.)

### Care Coach Backend Notifications:

Account Management

A registered user creates an account and is assigned to the Care Coach

A registered user becomes a member

RDF is utilized or requested

Billing Details

Credit Card Expiration date is within 30 days (notified on Jun. 1, 2021 if expires June 2021 )

Invoice is disputed via PayPal, credit card, etc.

Auto-renew is cancelled

Member Engagement

Quiz answers or scores are updated

Member gives a less than 5-star rating for anything

Communications

Vet Chat occurs (async—when initiated, or live—when concluded)

A member messages the Care Coach

A Care Coach response has not been replied to by the Member within 72 hours

Member Management

Member anniversary (notify 2 weeks in advance)

Member Birthday (notify 2 weeks in advance)

Pet Birthday (notify 2 weeks in advance)

As will be appreciated various other scenarios are possible and can be implemented on hardware and systems such as described herein.

In the above description of the present invention, reference is made to the accompanying drawings, which form a part hereof, and in which is shown by way of illustration specific embodiments in which the invention is may be practiced. It is understood that other embodiments may be utilized and structural changes may be made without departing from the scope of the present invention.

Embodiments of the present invention may comprise or utilize a special purpose or general-purpose computer including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, embodiments of the invention can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. RAM can also include solid state drives. Thus, it should be understood that computer storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Devices can have touch screens as well as other I/O components.

The described aspects can also be implemented in cloud computing environments. In this description and the following claims, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud computing model can be composed of various characteristics such as, for example, on-demand self-service, broad network access, resource pooling, rapid elasticity, measured service, and so forth. A cloud computing model can also expose various service models, such as, for example, Software as a Service (“SaaS”), Platform as a Service (“PaaS”), and Infrastructure as a Service (“IaaS”). A cloud computing model can also be deployed using different deployment models such as private cloud, community cloud, public cloud, hybrid cloud, and so forth. In this description and in the claims, a “cloud computing environment” is an environment in which cloud computing is employed.

Although the components and modules illustrated herein are shown and described in a particular arrangement, the arrangement of components and modules may be altered to process data in a different manner. In other embodiments, one or more additional components or modules may be added to the described systems, and one or more components or modules may be removed from the described systems. Alternate embodiments may combine two or more of the described components or modules into a single component or module.

The foregoing description has been presented for the purposes of illustration and description. It is not intended to be exhaustive or to limit the invention to the precise form disclosed. Many modifications and variations are possible in light of the above teaching. Further, it should be noted that any or all of the aforementioned alternate embodiments may be used in any combination desired to form additional hybrid embodiments of the invention.

Further, although specific embodiments of the invention have been described and illustrated, the invention is not to be limited to the specific forms or arrangements of parts so described and illustrated. The scope of the invention is to be defined by the claims appended hereto, any future claims submitted here and in different applications, and their equivalents.

What is claimed:

1. A method for providing pet related services to a user, the method comprising:
payment by the user into an accrual account;
evaluation of pet health by both a veterinarian and machine system;
confirmation of veterinary emergency visit by the user and pet; and
payment from the accrual account after confirmation of veterinary emergency visit.

2. The method for providing pet related services to a user of claim 1, wherein evaluation of pet health includes receiving information by an audio request on a cloud connected device supporting voice detection and voice response.

3. The method for providing pet related services to a user of claim 1, further comprising use of a cloud connected device that is a voice controlled virtual assistant.

4. The method for providing pet related services to a user of claim 1, further comprising user interactions with a machine script.

5. The method for providing pet related services to a user of claim 1, further comprising conversational user interactions that result in receipt of a product or service.

6. The method for providing pet related services to a user of claim 1, further comprising user interactions that result in a veterinary consult.

7. The method for providing pet related services to a user of claim 1, further comprising user interactions that result in interaction with other users;

8. The method for providing pet related services to a user of claim 1, further comprising short term safety monitoring of household pets.

9. The method for providing pet related services to a user of claim 1, further comprising long term health tracking.

10. The method for providing pet related services to a user of claim 1, further comprising providing supplementary information to the user.

11. A system supporting pet related services to a user, the system comprising:
a payment services system allowing payment by the user into an accrual account; and
a pet health evaluation system supporting interaction by a user with both a veterinarian and machine system, with the pet health evaluation system supporting confirmation of veterinary emergency visit by the user and pet and providing payment from the accrual account after confirmation of veterinary emergency visit.

12. The system for providing pet related services to a user of claim 11, wherein the pet health evaluation system receives information by an audio request on a cloud connected device supporting voice detection and voice response.

13. The system for providing pet related services to a user of claim 11, wherein the pet health evaluation system further includes a cloud connected device that is a voice controlled virtual assistant.

14. The system for providing pet related services to a user of claim 11, wherein the pet health evaluation system further includes support for user interactions with a machine script.

15. The system for providing pet related services to a user of claim 11, wherein the pet health evaluation system further includes conversational user interactions that result in receipt of a product or service.

16. The system for providing pet related services to a user of claim 11, wherein the pet health evaluation system further includes support for user interactions that result in a veterinary consult.

17. The system for providing pet related services to a user of claim 11, wherein the pet health evaluation system further includes support for user interactions that result in interaction with other users;

18. The system for providing pet related services to a user of claim 11, wherein the pet health evaluation system further includes support for short term safety monitoring of household pets.

19. The system for providing pet related services to a user of claim 11, wherein the pet health evaluation system further includes support for long term health tracking.

20. The system for providing pet related services to a user of claim 11, wherein the pet health evaluation system further includes support for providing supplementary information.

21. A method for providing pet related services to a user, the method comprising:
providing an intake form to the user after an audio request on a cloud connected device supporting voice detection and voice response or through an app;
providing evaluation of pet health by a machine system based at least in part on points or rankings recorded by the user in the intake form;
providing diagnosis or advisory information from the evaluation to a database and a veterinarian.

22. The method for providing pet related services to a user of claim 21, further comprising conversational user interactions that result in receipt of a product or service.

23. The method for providing pet related services to a user of claim 21, further comprising user interactions that result in a veterinary consult.

24. The method for providing pet related services to a user of claim 21, further comprising user interactions that result in interaction with other users;

25. The method for providing pet related services to a user of claim 21, further comprising short term safety monitoring of household pets.

26. The method for providing pet related services to a user of claim 21, further comprising long term health tracking.

27. The method for providing pet related services to a user of claim 21, further comprising providing supplementary information to the user.

28. The method for providing pet related services to a user of claim 21, further comprising payment from an accrual account after confirmation of veterinary emergency visit
